(19) United States
(12) Patent Application Publication
(10) Pub. No.: US 2026/0183296 A1
(43) Pub. Date: Jul. 2, 2026

(54) COMBINATION FOR USE IN BRAIN HEALTH AND INCREASING MENTAL PERFORMANCE

(71) Applicant: SOCIÉTÉ DES PRODUITS NESTLÉ S.A., Vevey (CH)

(72) Inventors: Aline BICHSEL, Yverdon (CH); Laura TROVO, Paudex (CH)

(21) Appl. No.: 19/130,633

(22) PCT Filed: Nov. 17, 2023
(86) PCT No.: PCT/EP2023/082164

§ 371 Date: May 16, 2025

(30) Foreign Application Priority Data
Nov. 17, 2022 (EP) 22208062.4

(51) Int. Cl. A61K 31/519; A61K 31/145; A61K 31/4415; A61K 31/714; A61P 39/06

(52) U.S. Cl. CPC A61K 31/519; A61K 31/145; A61K 31/4415; A61K 31/714; A61P 39/06

(57) ABSTRACT

The present invention relates to compositions comprising taurine and at least one of B vitamins, in particular Vitamin B9, for increasing glutathione levels in the brain and/or improving mental performance such as motivational performance, and/or the ability to remained focus and sustain attention, and/or the feeling of energy and vigor.

### FIELD OF THE INVENTION

The present invention relates to compositions comprising taurine and at least one of B vitamins, in particular Vitamin B9, for increasing glutathione levels in the brain and/or improving mental performance such as motivational performance, and/or the ability to remained focus and sustain attention, and/or the feeling of energy and vigor.

### BACKGROUND OF THE INVENTION

Glutathione (GSH) is an essential antioxidant used by the body to prevent cellular and tissue damage. It is involved in many fundamental metabolic processes ranging from the nitric oxide cycle to dietary mineral incorporation. Additionally, glutathione is instrumental for cells to regulate their division and their differentiation from progenitor cells into mature somatic cells.

As one of the body's core antioxidants, glutathione binds circulating reactive oxygen species (ROS) which can cause cellular and DNA damage if left unchecked. Reactive oxygen species, also known as free radicals, are byproducts of metabolism and can be broadly harmful to the body. To scavenge circulating ROS, glutathione binds to ROS thereby becoming oxidized. This means that glutathione prevents important cellular proteins or DNA from being oxidized, which can inhibit their function.

Mitochondria are known to be essential organelles that produce energy to facilitate cellular, physiological, and behavioral responses (Picard et al., 2018). The brain consumes approximately 20% of the body's total energy (Rettberg et al., 2014), and stress can amplify that need, leading to increased mitochondrial activity to meet energy demands (spare capacity) (Morava and Kozicz, 2013; Picard et al., 2018). Thus, mitochondria have been recently identified as vital for stress adaptation and cognitive functions (Fattal et al., 2006; Khacho M. et al., 2017; Picard et al, 2013).

Increased oxidative damage and decreased glutathione levels are observed under situations of high energetic demand in the brain such as psychogenic stress.

High concentrations of oxidized glutathione in the brain are a hallmark that the brain is in a compromised state, but high concentrations in the blood plasma may be considered to be healthy and normal. The reason is that oxidized glutathione must return to the bloodstream from the brain in order to discharge the ROS it carries into metabolic processes which can make use of them constructively. Alternatively, the oxidized form of GSH can be locally reverted back into the reduced state by glutathione reductase or it can return from the brain to the bloodstream in order to discharge the ROS constructively. As such, high concentrations of oxidized glutathione in the brain may mean that there is not enough glutathione to remove all of the reactive oxygen species that are circulating, indicating severe levels of stress.

By only measuring glutathione levels in blood plasma, one may erroneously assume that circulating glutathione is normal, even in cognitively impaired individuals. Only recently, it was recognized that cognitively impaired individuals have decreased glutathione levels in the brain, however, it is not known under what conditions glutathione levels in the brain may transiently change in normal healthy individuals related to their performance of different cognitive and motor tasks and motivation as it has been showed that high glutathione in the nucleus accumbens drives motivated behaviours in “Glutathione in the nucleus accumbens regulates motivation to exert reward-incentivized effort” Zalachoras et al. 2022 (https://www.ncbi.nlm.nih.gov/pmc/articles/PMC9642999/).

Direct supplementation with GSH is a challenging approach, particularly because it can be rapidly degraded in the liver into its constituent amino acids as well as be partially hydrolyzed and oxidized. Consequently, GSH bioavailability is limited following oral administration. Therefore, there is a need to find compositions and methods of increasing glutathione in the brain and sustaining antioxidant to support situations of high energetic demand in the brain.

### SUMMARY OF THE INVENTION

The present invention provides composition and method for enhancement of glutathione levels in the brain. In particular, the present invention provides composition comprising taurine and Vitamin B9 in a specific effective ratio. The present composition is for use in increasing glutathione levels in the brain and/or improving mental performance, in particular motivational performance, focus and/or sustained attention, fatigue in an individual.

As mentioned above, direct oral administration of glutathione has limited bioavailable effectiveness.

In a preferred embodiment, the composition is in the form of a feed, a beverage, a food supplement or a dietary supplement.

In a preferred embodiment, the composition further comprises at least one of B Vitamins selected from Vitamin B6 and/or Vitamin B12.

### DESCRIPTION OF FIGURES

FIG. 1: Comparative effect of taurine and different taurine to Vitamin B9 ratios on intracellular GSH synthesis

FIG. 2: Cross-sectional analyses of first period-data from motivation task (MIDT) over visits, predicted means±standard errors are presented. The timing between baseline and postintervention visit was 4 weeks.

FIG. 3a: Mitochondrial spare capacity (SPC) declines with oxidative stress.

FIG. 3b: Mitochondrial coupling efficiency (CE) decline with oxidative stress.

FIG. 4: effects of taurine, Vitamin B9 and compositions comprising taurine and Vitamin B9 in different ratios on mitochondria spare capacity (SPC).

FIG. 5: effects of taurine, Vitamin B9 and compositions comprising taurine and Vitamin B9 in different ratios on mitochondria coupling efficiency (CE).

### DETAILED DESCRIPTION OF THE INVENTION

### Definitions

All percentages are by weight of the total weight of the composition unless expressed otherwise. Similarly, all amounts and all ratios are by weight unless expressed otherwise. When reference is made to the pH, values correspond to pH measured at 25° C. with standard equipment. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used herein and in the appended claims, the singular form of a word includes the plural, unless the context clearly dictates otherwise. Thus, the references “a,” “an” and “the” are generally inclusive of the plurals of the respective terms.

Similarly, the words “comprise,” “comprises,” and “comprising” are to be interpreted inclusively rather than exclusively. Likewise, the terms “include,” “including” and “or” should all be construed to be inclusive, unless such a construction is clearly prohibited from the context. However, the embodiments provided by the present disclosure may lack any element that is not specifically disclosed herein. Thus, a disclosure of an embodiment defined using the term “comprising” is also a disclosure of embodiments “consisting essentially of” and “consisting of” the disclosed components.

Where used herein, the term “example,” particularly when followed by a listing of terms, is merely exemplary and illustrative, and should not be deemed to be exclusive or comprehensive. Any embodiment disclosed herein can be combined with any other embodiment disclosed herein unless explicitly indicated otherwise.

The “subject” or “individual” of the present invention is a human adult subject, preferably a healthy adult, an animal, a pet with the need to improve motivational and/or mental performance through modulating glutathione levels in the brain and/or protecting mitochondria from oxidative stress and guarantee adequate cellular energy. The compositions of the invention may be beneficially used for increasing glutathione level in the brain and/or protecting mitochondria from oxidative stress, for preventing or treating conditions or diseases which are characterized by low glutathione levels in the brain or mitochondrial dysfunction, whether transient or chronic.

“Reactive oxygen species” play important roles in cell signaling, a process termed redox signaling. Thus, to maintain proper cellular homeostasis a balance must be struck between reactive oxygen production and consumption. One source of reactive oxygen under normal conditions in humans is the leakage of activated oxygen from mitochondria during oxidative phosphorylation. Other enzymes capable of producing superoxide (O₂—) are xanthine oxidase, NADPH oxidases and cytochromes P450. Hydrogen peroxide, another strong oxidizing agent, is produced by a wide variety of enzymes including several oxidases.

The terms “treatment” and “treating” include any effect that results in the improvement of the condition or disorder, for example lessening, reducing, modulating, or eliminating the condition or disorder. The term does not necessarily imply that a subject is treated until total recovery. Non-limiting examples of “treating” or “treatment of” a condition or disorder include: (1) inhibiting the condition or disorder, i.e., arresting the development of the condition or disorder or its clinical symptoms and (2) relieving the condition or disorder, i.e., causing the temporary or permanent regression of the condition or disorder or its clinical symptoms. A treatment can be patient- or doctor-related.

The terms “prevention” or “preventing” mean causing the clinical symptoms of the referenced condition or disorder to not develop in an individual that may be exposed or predisposed to the condition or disorder but does not yet experience or display symptoms of the condition or disorder. The terms “condition” and “disorder” mean any disease, condition, symptom, or indication.

The relative terms “improved,” “increased,” “enhanced” and the like refer to the effects of the composition on increasing glutathione in the brain and subsequently improving the cognitive and/or motor performance in the individual subject.

“Mental performance” is conceptualized as the ability to remain motivated, focused, feeling vigorous and less fatigued, and is paramount for accomplishing daily tasks, fulfilling work responsibilities, and allowing people to actively engage in hobbies and leisure activities. Lapses in any of these domains can lead to decrements in mental performance, underscoring the need of the individuals to retain high levels of motivation/vigor and focus/sustained attention, as well as lowering levels of fatigue, to promote better overall performance. The link between focus, motivation, and fatigue, is well described and points to a bidirectional relationship among all these components (emotion-cognition-motivation interactions) (Pessoa, L., How do emotion and motivation direct executive control? Trends Cogn Sci, 2009. 13 (4): p. 160-6).

“Motivational performance” or “Motivation” is synonymous with the terms “mental energy” and related terms of “volition”, “will-power”, “time-on-task”, “persistence”, “self-control”, “sustained effort”, and “self-efficacy”, “effort perception”. All these terms relate to a person's drive to initiate and do things. Motivation is linked to subjectively perceived self-efficacy and well-being.

Motivational performance describes the subjective perception of mental resources available, subjective perception of “feeling of energy” and/or “feeling of vigor” which in turn is linked to cognitive functioning (Egan et al. (2015) Personality & Social Psychology Bulletin, 41 (3), 336-350). For example, motivational performance is reduced in states of depression and anxiety (O'Connor et al. (2006) Nutrition Reviews, 64 (7 Pt 2), S2-6).

“Motivational performance” or “motivation” can be measured by the performance in both motor tasks and cognitive tasks. Typically, these motor and cognitive tasks are performed under incentivized conditions, meaning that individuals get an incentive depending on their performance of the task.

For example, a motor task under incentivized conditions may be measured as an individual's ability to perform a strenuous motor task, e.g. squeezing a handgrip measuring both force and endurance wherein the performance is normalized for individual muscular strength (Zhu et al. (2019) Neurolmage. Clinical, 23, 101922; Berchio et al., 2019, Strasser et al. 2020).

For example, a cognitive task under incentivized conditions may be an individual's ability to perform a strenuous cognitive task, e.g. continuous/sustained attention and working memory (e.g. Unsworth et al. (2019) Journal of Experimental Psychology. Learning, Memory, and Cognition), mental arithmetic, or spatial reasoning (e.g. Nagase et al. (2015) Journal of the Society for Neuroscience, 38 (10), 2631-2651) wherein the performance is normalized for individual capacity to perform this task.

Motivation is a complex construct and is influenced both by internal and external sources, such as personal interests (i.e., internal) and anticipation of rewards or punishment (i.e., external). These different aspects of motivation can also be measured through validated self-reported questionnaires. For example, the Global Motivation Scale (GMS) is a scale designed to assess aspects of intrinsic motivation, extrinsic motivation, and amotivation to perform activities. As such, it can provide important insights into the motivation underlying participants' decision to engage with an activity. Additionally, the vigor subscale of the Profile of Mood States (POMS) can provide important insights into the level of perceived level of ‘resources’ that participants have at their disposal to perform mentally or physically challenging activities.

In animals, such as rodents, measurement of “motivational performance” is measured through motor tasks such as the forced swim test or cognitive tasks such as social dominance test or operant conditioning. Motivational performance can also be measured in relation to anxiety in tests such as “elevated plus maze” (e.g. Hollis et al. (2018) Neuropharmacology, 138, 245-256) and “open field and novel object” (e.g. Toledo-Rodriguez and Sandi, (2011) Frontiers in Behavioral Neuroscience, 5, 17).

“Focus” and/or “sustained attention” is the ability to remain focused and fully attentive on the task at hand. Among the different cognitive domains, the ability to retain focus and concentration when tasks require high levels of continuous attention is considered evidence of mental capacity. Specifically, performance declines in vigilance tasks, conceptualized as errors of omission/commission, are thought to be related to mental fatigue, and can provide a proxy of mental performance capacity. Among these vigilance tasks, the Psychomotor Vigilance Test (PVT) is often used to detect changes in sustained attention (focus/concentration)

“Fatigue” or levels of perceived fatigue is a main predictor of the capacity of people to perform demanding tasks. It's a state of weariness that reduces energy, motivation and concentration. Fatigue at this level impacts emotional and psychological well-being, Among the instruments measuring fatigue, the Profile Of Mood States (POMS) has been consistently used to assess fatigue in the context of nutritional interventions. The POMS is a questionnaire consisting of 65 items (37 items for POMS-short form) that load into 6 mood constructs: tension/anxiety, depression/dejection, anger/hostility, fatigue/inertia, confusion/bewilderment, and vigor/activity.

The “nucleus accumbens” is the most ventral part of the striatum and is mainly connected to the limbic system. As a functionally central structure between amygdala, basal ganglia, mesolimbic dopaminergic regions, mediodorsal thalamus and prefrontal cortex, the nucleus accumbens appears to play a modulative role in the flow of the information from the amygdaloid complex to these regions. Together with the prefrontal cortex and amygdala, the nucleus accumbens is part of the cerebral circuit which regulates functions associated with effort or motivated performance. It is anatomically located in a unique way to serve emotional and behavioral components of feelings. It is considered as a neural interface between motivation and action, having a key-role in food intake, sexual behavior, reward-motivated behavior, stress-related behavior and substance-dependence (Mavridis, Psychiatriki. 2015 October-Dec;25 (4): 282-94).

Mitochondrial function within the Nucleus accumbens and Pre-Frontal Cortex has a direct role in anxiety like and social behaviors (Hollis et al., 2015, 2018) as well as other brain regions (Burtscher J, et al., 2022) as well in “depressive-like behaviours” (Filipović D. et al., 2017; Ciubuc-Batcu M T t al., 2023) and age-related cognitive decline (Glavis-Bloom C. et al., 2023).

Importantly trait anxiety seems to negatively impact attentional control (Basten et. al., 2011; Berggren et al. 2013) and mind wandering (Fell J. et al., 2023), processing efficiency (Derakshan et al., 2009) and motivation due to changes in incentive processing when rewards require effortful exertion (Berchio et al. 2019). As well as depressive symptoms are characterized by low mood, lack of motivation, negative cognitive outlook, and sleep problems (Piao J, et al., 2023).

Moreover, a higher level of glutathione measured in the nucleus accumbens significantly correlates with improved motivational performance (https://www.ncbi.nlm.nih.gov/pmc/articles/PMC9642999/).

The terms “food,” “beverage”, “food product”, “beverage product”, “food composition” and “beverage composition” mean a product or composition that is intended for ingestion by an individual such as a human and provides at least one nutrient to the individual. The compositions of the present disclosure, including the many embodiments described herein, can comprise, consist of, or consist essentially of the essential elements and limitations described herein, as well as any additional or optional ingredients, components, or limitations described herein or otherwise useful in a diet.

The composition can be any kind of composition that is suitable for human and/or animal consumption. For example, the composition may be selected from the group consisting of: food compositions, dietary supplements, nutritional compositions, nutraceuticals, powdered nutritional products to be reconstituted in water or milk before consumption, food additives, medicaments, beverages and drinks, including coffee based products (coffee RTD, coffee capsules, etc.,). In an embodiment, the composition is an oral nutritional supplement (ONS), a complete nutritional formula, a pharmaceutical, a medical or a food product. In a preferred embodiment, the composition is administered to the individual as a beverage. The composition may be stored in a sachet as a powder and then suspended in a liquid such as water for use.

As used herein, “complete nutrition” contains sufficient types and levels of macronutrients (protein, fats and carbohydrates) and micronutrients to be sufficient to be a sole source of nutrition for the individual to which the composition is administered. Individuals can receive 100% of their nutritional requirements from such complete nutritional compositions.

Administration of the compositions of the invention encompass “enteral administration” in all forms, although oral administration is preferred.

Each of the compounds can be administered at the same time as the other compounds (i.e., as a single unit) or separated by a time interval (i.e., in separate units).

All modes of administration may be considered in combination with glutathione per se.

### EMBODIMENTS

The composition comprises taurine: Vitamin B9 in a ratio of about 1:2500. It is for use in increasing glutathione levels in the brain and/or improving mental performance, motivational performance, focus, sustained attention and/or fatigue in an individual.

Such composition increases glutathione in the nucleus accumbens region of the brain to provide the benefits to the subject.

### Glutathione

Glutathione (GSH) is the most abundant intracellular component of overall antioxidant defenses. GSH, a tripeptide, is synthesized from precursor amino-acids: glycine, cysteine and glutamate in two steps catalyzed by glutamate cysteine ligase (GCL, also known as gamma-glutamylcysteine synthetase, EC 6.3.2.2) and gamma-L-glutamyl-L-cysteine: glycine ligase (also known as glutathione synthetase, EC 6.3.2.3), and GSH synthesis occurs de novo in cells.

Glutathione is also known as Gamma-Glutamylcysteinylglycine, Gamma-L-Glutamyl-L-Cysteinylglycine, Gamma-L-Glutamyl-L-Cysteinylglycine, Glutathion, Glutation, L-Gamma-Glutamyl-L-Cysteinyl-Glycine, L-Gamma-Glutamyl-L-Cysteinyl-Glycine, L-Glutathion, L-Glutathione, GSH, N-(N-L-gamma-Glutamyl-L-cysteinyl)glycine. It is typically administered as S-acetyl glutathione or reduced L-glutathione.

Glutathione-rich food include: cruciferous vegetables, for example, broccoli, cauliflower, Brussels sprouts, and bok choy; allium vegetables, for example, garlic and onions; eggs, nuts, legumes, lean protein, such as fish, and chicken as well as whey protein. Glutathione-rich herbs include: for example, milk thistle, flaxseed, guso seaweed. Compositions and methods of the invention can also be used in combination with dietary recommendations for a glutathione-rich food to complement the diet.

Lifestyle parameters may affect levels of glutathione in the brain. For example, glutathione is also negatively affected by insomnia. Therefore, compositions and methods of the invention would also include the recommendation to have sufficient sleep.

Psychogenic stress is defined as a state of imminent or perceived threat to homeostasis, where the brain and body invoke various physiological responses to adapt. Glutathione levels in the brain may be affected by such stress.

### Taurine

Taurine also known as 2-aminoethanesulfonic acid is an organic acid that occurs naturally in food, especially in shellfish (eg, scallops, mussels, clams) and in the dark meat of turkey and chicken, as well as in other meats and eggs.

In a preferred embodiment the daily amount of taurine is administered to a human in an amount from at least about 500 mg.

In a particular non-limiting example, the daily doses for a human 60 kg subject are from about 500 mg to about 3000 mg per day of taurine or a functional derivative thereof.

In another embodiment the daily amount of taurine is administered to an animal or a pet in an amount of the equivalent conversion from the human dose to the animal dose based on body surface, as per FDA guidelines (Nair A B, Jacob S. A simple practice guide for dose conversion between animals and human. J Basic Clin Pharm. 2016 March;7 (2): 27-31. doi: 10.4103/0976-0105.177703. PMID: 27057123; PMCID: PMC4804402).

In an embodiment, the daily amount of taurine is administered to a dog in an amount at least about 15 mg per kg of body weight

In an embodiment, taurine and Vitamin B9 are comprised in ratio so that the respective amounts of Taurine and B9 in the composition are about 100 mg and 0.04 mg, or 120 mg and 0.05 mg, or 150 mg and 0.06 mg, or 200 mg and 0.08 mg, or 300 mg and 0.12 mg, or 400 mg and 0.16 mg, or 500 mg and 0.2 mg, or 600 mg and 0.24 mg, or 800 mg and 0.32 mg, or 1000 mg and 0.4 mg, or 2000 mg and 0.8 mg, or 3000 mg and 1.2 mg, for example.

Vitamin B9, also known as folate or folic acid, is not produced in the body, making it an essential nutrient. Folate naturally occurs in vegetables (especially dark green leafy vegetables), fruits and fruit juices, nuts, beans, peas, seafood, eggs, dairy products, meat, poultry, and grains.

The inventors surprisingly found the most effective amount of taurine as well as its ratio to Vitamin B9 in the composition for intracellular GSH increase.

Thus, in the first embodiment of the invention, composition comprises Vitamin B9 and taurine in the ratio of about 1:2500, meaning the amount of taurine is 2500 times more than the amount of Vitamin B9 in the composition.

In an embodiment, Vitamin B9 is in the amount of from about 0.2 mg to about 1.2 mg.

In an embodiment, the composition further comprises at least one of B Vitamins, preferably selected from Vitamin B6 and/or Vitamin B12.

### Other B Vitamins

One or more B vitamins are used in the composition. Non-limiting examples of suitable B vitamins include Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin) and Vitamin B12 (various cobalamins; commonly cyanocobalamin in vitamin supplements) and combinations thereof. “Vitamin” includes such compounds obtained naturally from plant and animal foods or synthetically made, pro-vitamins, derivatives thereof, and analogs thereof.

In several embodiments the composition is for use in increasing glutathione levels in the brain and/or improving mental performance, in particular motivational performance, focus and/or sustained attention, fatigue.

In an embodiment, the composition to be administered to a human further comprises at least one of
Vitamin B9 in the amount of from about 0.2 mg to about 1.2 mg,
Vitamin B6 in the amount of from about 1.3 mg to about 7.8 mg,
Vitamin B12 in the amount of from about 2.4 mcg to about 14.4 mcg.

In an embodiment the composition to be administered to an animal or a pet further comprises at least one of Vitamins B in an amount of the equivalent conversion from the human dose to the animal dose based on body surface, as per FDA guidelines.

In an embodiment the composition to be administered to a dog further comprises at least one of

Vitamin B9 in the amount of about 0.006 mg per kg of body weight, or the equivalent conversion from the human dose to the animal dose based on body surface, as per FDA guidelines,

Vitamin B6 in the amount of about 0.04 mg per kg of body weight, or the equivalent conversion from the human dose to the animal dose based on body surface, as per FDA guidelines,

Vitamin B12 in the amount of about 0.072 mcg per kg of body weight, or the equivalent conversion from the human dose to the animal dose based on body surface, as per FDA guidelines.

In an embodiment, the composition is administered orally.

In an embodiment, the composition is a food, a beverage, a food supplement or a dietary supplement.

In an embodiment, the composition is in the form of an RTD, an RTD coffee, a coffee capsule, a tablet, a gummy or powder sachet.

In an embodiment the invention relates to a use of the composition as disclosed herein in healthy individuals in need of at least one of i) improving motivational performance; ii) improving focus and/or sustained attention; iii) reducing fatigue.

In an embodiment, the composition is for use in improving cytoprotection and oxidative stress.

### Composition Formulations

In one embodiment, the compositions are food compositions, including human and pet food compositions. In several embodiments, the food composition is a product with at least one nutrient for improving motivation performance or mental energy.

For pet food compositions, they may supply the necessary dietary requirements for an animal, animal treats (e.g., biscuits), or dietary supplements. The compositions may be a dry composition (e.g., kibble), semi-moist composition, wet composition, or any mixture thereof. In another embodiment, the composition is a dietary supplement such as a gravy, drinking water, beverage, yogurt, powder, granule, paste, suspension, chew, morsel, treat, snack, pellet, pill, capsule, tablet, or any other suitable delivery form. The dietary supplement is to be administered to the animal in small amounts, or in the alternative, can be diluted before administration to an animal. The dietary supplement may require admixing or can be admixed with water or other diluent prior to administration to the animal.

In another embodiment, the composition is in the form of a food supplement or dietary supplement, in particular in the form of a tablet, a powder sachet or a gummy.

In a preferred embodiment, the composition is in the form of an effervescent tablet.

The effervescent tablets can be produced and controlled same as conventional tablets. These controls include physicochemical properties such as hardness, weight variation, friability, solution time, pH and content uniformity. The effervescent tablets can be produced by a direct compression method, a fusion method, a wet or dry granulation method, or any other suitable method. Low relative humidity (e.g., maximum of 25% or less) and moderate to cool temperatures (e.g., about 25° C. or 77° F.) in the environment may be essential to prevent sticking granule or tablets to the tablet press machine.

In the direct compression method, the effervescent tablet can be formed by compressing the ingredients in the form of powders into a dense mass, for example, by a tablet press machine. The powdered ingredients may be first granulized to similar or equal sizes before being made into tablets, so that the mixtures of powder have excellent flowability without particles segregation. Granulating may not be required if the raw materials are selected to achieve a free-flowing, non-segregating, compressible powder blend. The tablets can then be dried by heat, such as in an oven with air circulation, at a suitable temperature for a suitable time and after cooling can be packed in a suitable package.

In the fusion method, the ingredients can be mixed in a suitable mixer, such as a blender, for an appropriate time. Then, the obtained mixture can be heated to a suitable temperature. The powder may be mixed regularly until the crystallization water of citric acid is released as binder factor (e.g., approximately 30 minutes) and an appropriate pasty mass is obtained. This wet mass can be passed through a sieve to obtain the desired granules, which can then be dried at a suitable temperature for an appropriate time. After drying, the granules can be passed through the sieve again. Other ingredients can be added to the granule mass and mixed for a suitable time. The granule mixtures then can be compressed into tablets by the tablet press machine. Finally, the tablets can be dried and packed in a suitable package.

In the wet granulation method, the ingredients may be milled by a miller, either separately or as a mixture with ethanol, ethanol-water mixture, isopropanol, etc., and the obtained powder can be passed through a sieve and then blended. A binder solution can be added to the mixture to form a pasty mass. This pasty mass can then be passed through a sieve to obtain desired granules, which can then dried. The dried mass can be passed through a sieve again, and other ingredients can be added and mixed. The obtained granule mixtures then can be compressed into tablets by the tablet press machine. Finally, the tablets can be dried and packed in a suitable package. Wet granulation can also be performed by carefully adding 0.1 to 1.0 percent water (weight-to-weight basis) to a blend of raw materials that possess the uniformity, compressibility, and flowability needed to produce good-quality tablets, but which lacks the needed binding properties. The free water which is usually added in the form of a fine spray to selected formulation components while mixing in a suitable blender acts as a binder. The granulation steps must be precisely timed and the ingredients mixed thoroughly to distribute the granulating fluid evenly in the blend. The mix is then quickly discharged to drying ovens. After drying, the granules are sized, and a final mix is performed. The granules are then compressed into tablets using tablet machines.

The dry granulation method can use special processing equipment known as a “roller compactor” or “chilsonator.” These machines compress premixed powders between two counterrotating rollers under extreme pressure. Depending on the configuration of the roller, the feed material may be compacted into dense ribbon-like materials known as flakes (smooth rolls) or dense briquettes (almond or stick-shaped) if the rollers have grooved or etched surfaces. The compressed material is reduced to the proper size for tablet granulation purposes. Another dry granulation procedure is slugging, in which the powder particles are compressed into large flat tablets or pellets using a tablet press or more usually, a heavy-duty tablet-compacting equipment. The resulting tablet or slug are milled to yield the desired granule characteristics.

The effervescent tablet can be made in any shape and can have any suitable size. As non-limiting examples, the tablet can be 5 mm to 20 mm long, thick, and/or in diameter if having a round shape. The size of the tablet can be from 5 mm to 10 mm, 5 mm to 15 mm, 10 mm to 15 mm, 10 mm to 20 mm, or 15 mm to 20 mm.

The effervescent tablet may comprise a binder, such as polyvinylpyrolidone (PVP) or any other suitable binder. The binder is preferably water-soluble. It can be added as dry powder or in a wet form as an aqueous or hydroalcoholic solution. Mannitol, PEG 6000 and water in small amounts can also be used as a binder. PEG 6000 at 3% use level can be used as a dry binder. The ideal amount of binder is one that makes the tablet hard enough to handle but soft enough to disintegrate and dry enough to be stable. The effervescent tablet may also be formulated without a binder.

### Beverage Compositions

In one embodiment, the compositions are beverage compositions. Such beverage compositions are meant to be consumed by a human or animal. In several embodiments, the beverage is a milk-based beverage; a performance nutrition product, a medical nutrition product; a milk product, e.g. a milk drink, a product with at least one nutrient for improving motivation performance or mental energy.

### Dairy Product

In one embodiment, the composition can be formulated as a “dairy product” together with milk proteins, e.g., milk protein concentrate or milk protein isolate; caseinates or casein, e.g., micellar casein concentrate or micellar casein isolate; or whey protein, e.g., whey protein concentrate or whey protein isolate. Additionally, or alternatively, at least a portion of the protein can be plant protein such as one or more of soy protein, pea protein or canola protein.

### Nutritional Supplement

In one embodiment, the composition of the invention can be formulated as a “nutritional supplement” together with glutathione enhancing compounds of the invention. The compounds of the invention can be used alone or in combination with appropriate additives to make tablets, gummies, powders, granules or capsules, for example, with conventional additives, such as lactose, mannitol, corn starch or potato starch; with binders, such as crystalline cellulose, cellulose functional derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

### Administration

The composition of the invention can be administered at least one day per week, preferably at least two days per week, more preferably at least three or four days per week (e.g., every other day), most preferably at least five days per week, six days per week, or seven days per week. The time period of administration can be at least one week, preferably at least one month, more preferably at least two months, most preferably at least three months, for example at least four months. In an embodiment, dosing is at least daily; for example, a subject may receive one or more doses daily. In some embodiments, the administration continues for the remaining life of the individual. In other embodiments, the administration occurs until no detectable symptoms of the condition remain. In specific embodiments, the administration occurs until a detectable improvement of at least one symptom occurs and, in further cases, continues to remain ameliorated.

The ideal duration of the administration of the composition can be determined by those of skill in the art.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

### EXAMPLES

### Example 1

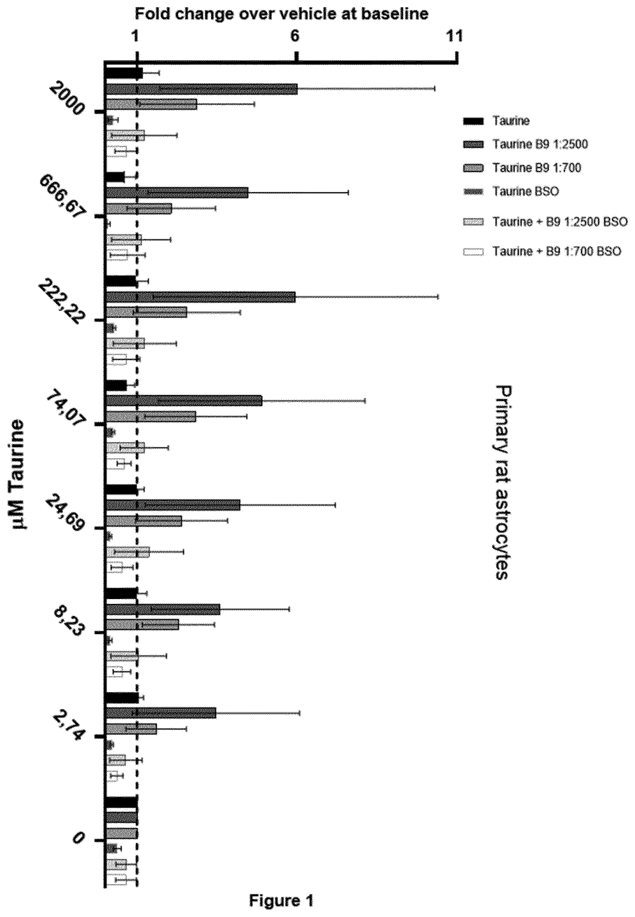

The inventors investigated the effect of different taurine to Vitamin B9 ratios on intracellular glutathione (GSH) synthesis (FIG. 1). To measure GSH increase after taurine and Vitamin B9 supplementation at different ratios, rat primary astrocytes were cultured in medium depleted of vitamin B9, but still supplemented with 15% FBS to not have a full depletion but rather a reduction of this vitamin, as it would be more physiologically relevant for human applications.

Because standard DMEM is very high in vitamin B9 (about 9 μM), to see a full dynamic range in response to the supplementation the inventors used a reduction in such vitamin and not a total depletion as this would not reflect a human status.

The following amounts and ratios of the ingredients of the composition were used (table 1):

TABLE 1
Taurine and Vitamin B9 amounts and ratios used in comparative
analysis for the effect on intracellular GSH increase

| | | Ratio 1:2500 | Ratio 1:700 |
|---|---|---|---|
| | Taurine (μM) | Vit B9 (μM) | Vit B9 (μM) |
| | 2000 | 0.8 | 2.9 |
| | 667 | 0.27 | 0.95 |
| | 222 | 0.09 | 0.32 |
| | 74 | 0.03 | 0.11 |
| | 25 | 0.01 | 0.04 |
| | 8 | 0.003 | 0.012 |
| | 3 | 0.001 | 0.004 |
| Vehicle (solvent) | 0 | 0 | 0 |

Intracellular GSH was measured in rat primary astrocytes in culture by using the GSH-Glo™ Assay, which is a luminescent-based assay for the detection and quantification of total glutathione levels in cells. The assay is based on the conversion of a luciferin derivative into luciferin in the presence of GSH. The reaction is catalyzed by a glutathione S-transferase (GST) enzyme supplied in the kit. The luciferin formed is detected in a coupled reaction using Ultra-Glo™ Recombinant Luciferase that generates a glow type luminescence that is proportional to the amount of glutathione present in cells. A standard curve is used for each biological replicate and at least 5 technical replicates were done for each condition and for each biological replicate (3 biological replicates). 10% of lysate of each technical and biological replicate is used to measure protein amount by BCA and used to normalize the GSH intracellular content to total protein amount. Buthionine sulfoximine (BSO) a specific inhibitor of γ-glutamylcysteine ligase (GCL) was added at 15 mM at the time of the start of the treatment to confirm the specificity of the readout.

Measurements were performed 48 hours after treatment. Results were compared to the control condition (vehicle) for each biological replicate and for each condition.

The inventors found the most effective taurine ratio to Vitamin B9 (1:2500) in the composition for intracellular GSH increase.

FIG. 1 shows the most effect on GSH increase of the composition taurine and its ratio to Vitamin B9 in the composition 1:2500

### Example 2

The effect of a 4-week oral supplementation of a combination of B vitamins (B6, B9, B12) and taurine vs Placebo was evaluated on motivation in healthy adults.

### Investigational Product

The investigational product (IP) was provided in single capsules, each containing
Taurine: 500 mg
Vitamin B6: 1.3 mg
Vitamin B9: 0.2 mg
Vitamin B12: 2.4 mcg

Placebo: the placebo consisted of identical looking capsules containing microcrystalline cellulose.

Both the IP and placebo were administrated to 44 participants (21 females and 23 males, 25-40 years of age) orally once daily in capsule form, ideally between conventional mealtimes (i.e. breakfast, lunch, supper), approximately 30 minutes to 1 hour before behavioral testing planned to commence.

Primary outcome is monetary incentive delay task (MIDT). Improved motivation was assessed by MIDT in changes from baseline to day 28 of product intake with an anticipated effect of 0.08. 1 MIDT trials start with the participant seated facing a laptop screen and holding a hand dynamometer connected to a BIOPAC system which records the force and grip duration. The screen will then show a fixation cross, followed by an anticipatory signal indicating the trial's incentive (e.g. 50; 20; 10 pesos). The beginning of the force exertion period is signaled by the appearance of a red circle around the fixation cross. If the established force threshold is reached within the specific time frame, the red circle is replaced by a green circle. The green circle also indicates that participants must maintain the contraction force level for a specific time. If participants did not reach the threshold in the initial time frame or if the force level fell below the threshold during maintenance period, the trial is failed, and visualized by a red cross occurring on the screen. If the force was maintained for the required time, a green tick indicates a successful task performance during a single trial. The task runs in blocks with a 3 min break between the blocks. Each block contains sessions and each session have trials: equal incentivized trial numbers of each of the different incentives in a random order and non-incentivized rest trials interspersed within every 3 incentivized trials. This test of motivation is expected to last for approximately 15-18 minutes.

The trial has a cross-over design, where a baseline and two follow-up measurements are crossing over, thus one can estimate a period effect or a carry-over effect comparing the two baselines. The timing between baseline and follow-up visit was 4 weeks. The washout period was also 4 weeks. We identified a period-effect and a carry-over effect which motivates analyzing only first-period-data cross-sectionally. Further we hypothesized effect modification by incentive, thus only the condition under incentives (10, 20, 50 pesos) was analyzed.

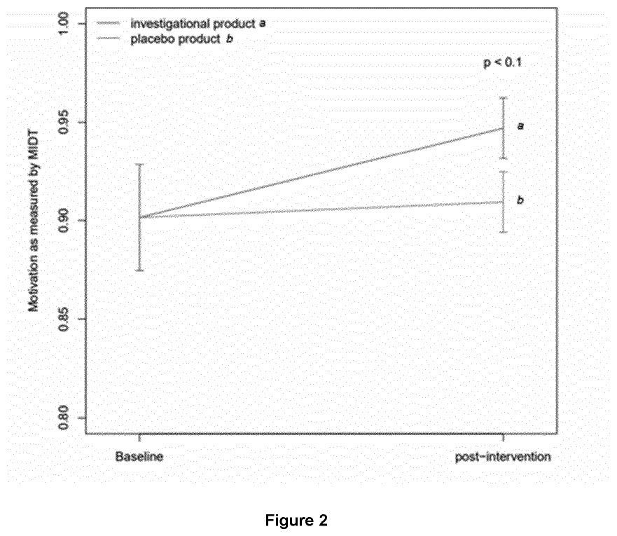

As it is demonstrated in the FIG. 2, the cross-sectional analysis shows an increase from baseline of 0.04 in MIDT as a statistical trend (p<0.1), indicting a trend in improved motivation in the investigational product condition compared to control.

### Example 3

Oxidative stress (induced by tert-Butyl Hydroperoxide (tBHP)) dose response to astrocytes and mitochondrial function were investigated.

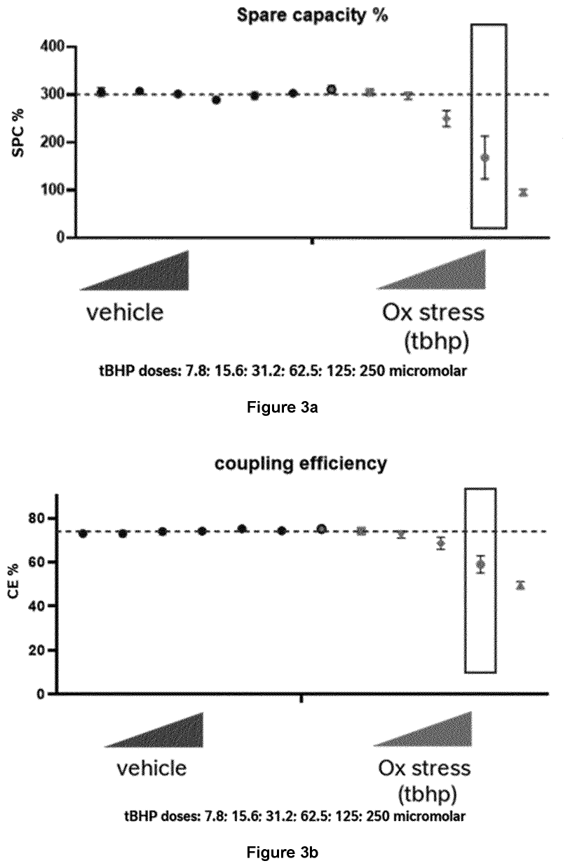

Primary astrocytes were cultured on Seahorse XF-96 (Seahorse BioSciences-Bucher Biotec AG) plates coated with poly-D-lysine: cat N° A3890401_Gibco at 0.1 mg/ml diluted at 50 ug/ml with phosphate-buffered saline (PBS), at a density of 15 000 cells per well. The cells were cultured in the medium described before and treated 48 hours before the oxidative stress application and metabolic flux analysis with Vitamin B9, taurine and compositions comprising taurie: Vitamin B9 in ratios 1:2500 and 1:1700. After performing a dose response of tBHP for 1 hour (7.8; 15.6; 31.2; 62.5; 125; 250 micromolar), see FIG. 3a and FIG. 3b, 125 μM tBHP was chosen to impact mitochondrial function and test the protective effects of the mentioned compounds.

As it is shown in FIG. 3a, mitochondrial function such as spare capacity declines with oxidative stress.

Mitochondrial metabolism must constantly adapt to stress or demanding conditions in order to maintain bioenergetic levels related to cellular functions. Metabolic adaptation calls on mitochondrial spare capacity to meet increasing needs.

As it is shown in FIG. 3b, mitochondrial function such as coupling efficiency declines with oxidative stress.

Tightly coupled mitochondria generate more ATP with less substrate and heat dispersion, while loosely coupled mitochondria generate less ATP with more substrate and heat dispersion, due to proton leakage.

Using the Seahorse XF metabolic analyzer we performed a “mitochondrial stress test” as per standard design (Gu X., et al, 2021), three baseline measurements of oxygen consumption rate (OCR) were sampled prior to sequential injection of mitochondrial inhibitors. Three metabolic determinations were sampled following addition of each mitochondrial inhibitor prior to injection of the subsequent inhibitors. The mitochondrial inhibitors used were oligomycin (1.5 μm), FCCP (carbonyl cyanide 4-(trifluoromethoxy)-phenylhydrazone) (3 μm), and antimycin (5 μm) and rotenone (0.5 μm). OCR was automatically calculated and recorded by the Seahorse software. After the assays, cells were stained with DAPI to perform a nuclear counting for normalization using ImageXpress Micro Confocal High-content Imaging system from Molecular Devices (OCR values/cell). The spare respiratory capacity is calculated by the Seahorse assay as (Maximal Respiration)/(Basal Respiration)×100, and coupling efficiency is calculated as ATP Production Rate)/(Basal Respiration Rate)×100. In the treatment assays the values are reported normalized to vehicles.

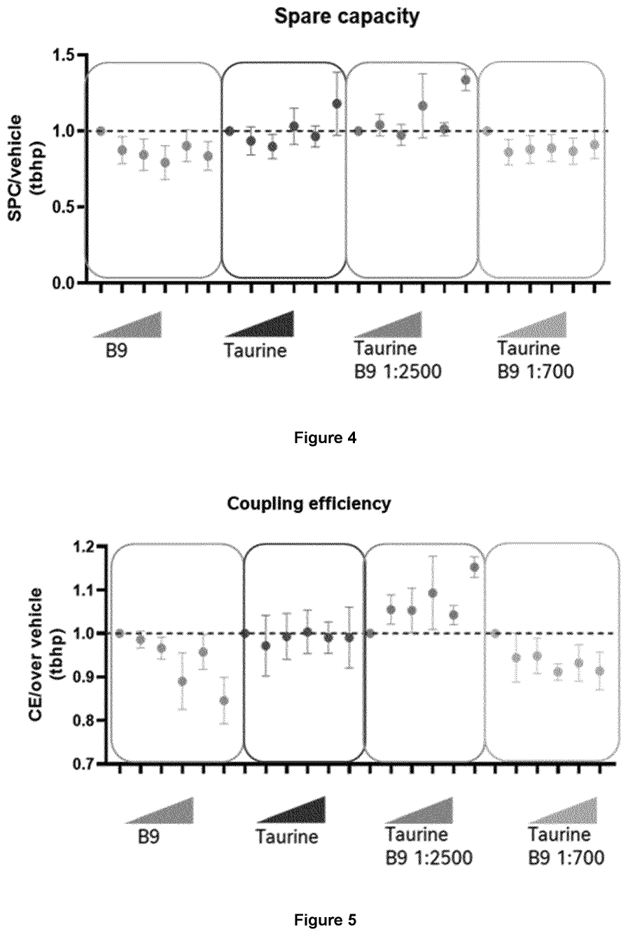

As demonstrated in FIG. 4, ratio taurine:Vitamin B9=1:2500 protects mitochondria from oxidative stress, by improving their spare capacity. Indeed, the other compounds alone or at the different ratio (1:700) make things worse than vehicle, while the ratio 1:2500 is showing a better effect and outperforming the others until the highest dose. Spare capacity is a key feature of mitochondria to adjust to cell energy demands.

FIG. 5 shows that ratio taurine:Vitamin B9 being about 1:2500 protects mitochondria from oxidative stress. Indeed, it increases the coupling efficiency in a dose response manner, which in turn supports the maximum production of ATP with lower energy dispersion.

1. A composition comprising taurine: Vitamin B9 in a ratio of about 1:2500.

2. A method of increasing glutathione levels in the brain and/or improving mental performance in an individual, the method comprising administering a composition comprising taurine: Vitamin B9 in a ratio of about 1:2500 to the individual.

3. A method of improving motivational performance, focus and/or sustained attention, and/or preventing fatigue in an individual, the method comprising administering a composition comprising taurine: Vitamin B9 in a ratio of about 1:2500 to the individual.

4. The method of claim 2, wherein the composition further comprises at least one additional B Vitamin.

5. The method of claim 2, wherein the composition further comprises Vitamin B6 and/or Vitamin B12.

6. The method of claim 2, wherein the respective amounts of the taurine and the Vitamin B9 in the composition are about 100 mg and 0.04 mg, or 120 mg and 0.05 mg, or 150 mg and 0.06 mg, or 200 mg and 0.08 mg, or 300 mg and 0.12 mg, or 400 mg and 0.16 mg, or 500 mg and 0.2 mg, or 600 mg and 0.24 mg, or 800 mg and 0.32 mg, or 1000 mg and 0.4 mg, or 2000 mg and 0.8 mg, or 3000 mg and 1.2 mg.

7. The method of claim 2, wherein the individual is a human, and the taurine is administered to the human in an amount of at least about 500 mg.

8. The method of claim 2, wherein the composition further comprises at least one of
Vitamin B9 in an amount from about 0.2 mg to about 1.2 mg,
Vitamin B6 in an amount from about 1.3 mg to about 7.8 mg, or
Vitamin B12 in an amount from about 2.4 mcg to about 14.4 mcg.

9. The method of claim 2, wherein the individual is an animal or pet, and the taurine and/or the Vitamin B9 is administered to the animal or pet in an amount of the equivalent conversion from the human dose to the animal dose based on body surface, as per FDA guidelines.

10. The method of claim 2, wherein the composition is a food composition, dietary supplement, nutritional composition, nutraceutical, powdered nutritional product to be reconstituted in water or milk before consumption, food additive, medicament, beverage and drink, oral nutritional supplement (ONS), a complete nutritional formula, a pharmaceutical, or a medical or a food product.

11. The method of claim 2, wherein the individual is a healthy individual in need of at least one of i) improving motivational performance; ii) improving focus and/or sustained attention;
or iii) reducing fatigue.

12. A method of improving cytoprotection and oxidative stress in an individual, the method comprising administering a composition comprising taurine: Vitamin B9 in a ratio of about 1:2500 to the individual.

13. The method of claim 3, wherein the composition further comprises at least one additional B Vitamin.

14. The method of claim 3, wherein the composition further comprises Vitamin B6 and/or Vitamin B12.

15. The method of claim 3, wherein the respective amounts of the taurine and the Vitamin B9 in the composition are about 100 mg and 0.04 mg, or 120 mg and 0.05 mg, or 150 mg and 0.06 mg, or 200 mg and 0.08 mg, or 300 mg and 0.12 mg, or 400 mg and 0.16 mg, or 500 mg and 0.2 mg, or 600 mg and 0.24 mg, or 800 mg and 0.32 mg, or 1000 mg and 0.4 mg, or 2000 mg and 0.8 mg, or 3000 mg and 1.2 mg.

16. The method of claim 3, wherein the individual is a human, and the taurine is administered to the human in an amount of at least about 500 mg.

17. The method of claim 3, wherein the composition further comprises at least one of
Vitamin B9 in an amount from about 0.2 mg to about 1.2 mg,
Vitamin B6 in an amount from about 1.3 mg to about 7.8 mg, or
Vitamin B12 in an amount from about 2.4 mcg to about 14.4 mcg.

18. The method of claim 3, wherein the individual is an animal or pet, and the taurine and/or the Vitamin B9 is administered to the animal or pet in an amount of the equivalent conversion from the human dose to the animal dose based on body surface, as per FDA guidelines.

19. The method of claim 3, wherein the composition is a food composition, dietary supplement, nutritional composition, nutraceutical, powdered nutritional product to be reconstituted in water or milk before consumption, food additive, medicament, beverage and drink, oral nutritional supplement (ONS), a complete nutritional formula, a pharmaceutical, or a medical or a food product.

20. The method of claim 3, wherein the individual is a healthy individual in need of at least one of i) improving motivational performance; ii) improving focus and/or sustained attention; or iii) reducing fatigue.
